(19) United States
(12) Patent Application Publication
(10) Pub. No.: US 2011/0244416 A1
(43) Pub. Date: Oct. 6, 2011

(54) APPARATUS, METHOD AND SYSTEM FOR DENTAL IMPLANTS

(76) Inventor: James Henderson Smith WHITNEY, Harrisonburg, VA (US)

(21) Appl. No.: 13/149,372

(22) Filed: May 31, 2011

Related U.S. Application Data
(63) Continuation-in-part of application No. 12/840,494, filed on Jul. 21, 2010.
Application No. 12/840,494 is a continuation-in-part of application No. 12/753,203, filed on Apr. 2, 2010.

(51) Int. Cl. A61C 8/00; A61B 6/14

(52) U.S. Cl. 433/29; 433/175

(57) ABSTRACT

A dental implant that can have a number of manners of being inserted and seated. The dental implant can include a base or platform coupled to any number of roots projecting there from which can be used to seat the implant in the mouth of a patient.

### CROSS-REFERENCE TO RELATED APPLICATIONS

The patent application is a continuation-in-part of pending U.S. patent application Ser. No. 12/840,494, filed Jul. 21, 2010 and U.S. patent application Ser. No. 12/753,203, filed on Apr. 2, 2010, the contents of which are incorporated by referenced in their entirety herein.

### BACKGROUND

Dental implants and orthopedic prostheses are commonly used in procedures to replace teeth that have been removed or which are missing. Typically implants are inserted into a hole disposed in the maxilla or mandible of a patient receiving the implant. The hole is commonly bored into the bone tissue of the maxilla or mandible through the use of a drill and the implant is inserted into the hole. In some situations, a hole bored into bone tissue can include some form of threading. The threading on an internal portion of the bored hole can then allow for a standard dental implant to be inserted into the hole and at least partially fixed into position. In other situations, implants may have a self-tapping or securing component, such as one or more projections, that allow the implant to be secured.

Implants of the types described above are commonly used in both anterior and posterior portions of the maxilla and mandible. Additionally, implants typically have a single root or securing protrusion, regardless of the ultimate location of the implant with the mouth of a patient. For example an implant having the structure described above may be implanted in any part of a mouth and may include a tooth portion that resembles any desired natural tooth.

Current dental implants and orthopedic prostheses, however, rely on a single cylinder which is implanted into the bone. As described above, the single cylinder functions as the root of the implant, allowing it to be implanted and secured into the bone of a patient's mouth. Single cylinder implants, however, rely on the single cylinder to absorb any pressure associated with the implant as well as being the lone source of securing the implant in a bone. Thus such implants often fail as aspects of the single cylinder and implant do not have the capability and strength to remain in a desired position when dealing with such forces. Such single cylinder implants are particularly prone to failure in posterior sections of the maxilla and mandible where the bone tissue is softer, accommodating the higher forces associated with use of teeth in those regions of the mouth.

### SUMMARY

Some exemplary embodiments include a dental implant that can have a number of manners of being inserted and seated. The dental implant can include an artificial crown and an abutment coupled to the implant. The platform portion of the implant may then have any number of roots projecting there from which can be used to seat the implant in the mouth of a patient.

Another exemplary embodiment can describe a system for a dental implant. The system can have an artificial crown, an abutment coupled to the implant, which can have a plurality of roots coupled to the platform to secure a dental implant.

### BRIEF DESCRIPTION OF THE DRAWINGS

FIG. 1A is an exemplary diagram of a front view of a dental implant.

FIG. 1B is an exemplary diagram of a side view of a dental implant.

FIG. 2A is an exemplary diagram of a front view of a dental implant.

FIG. 2B is an exemplary diagram of a side view of a dental implant.

FIG. 2C is an exemplary perspective view of a dental implant.

FIG. 3A is an exemplary top-down view of a dental implant.

FIG. 3B is an exemplary rotated top-down view of a dental implant.

FIG. 4 is an exemplary diagram of a platform router bit.

FIG. 5A is an exemplary front view of a saw.

FIG. 5B is an exemplary side view of a saw.

FIG. 6A is an exemplary front view of another saw.

FIG. 6B is an exemplary side view of another saw.

FIG. 6C is an exemplary front view of another saw.

FIG. 6D is an exemplary side view of another saw.

FIG. 7A is an exemplary diagram of a blade guide.

FIG. 7B is an exemplary diagram of another blade guide.

FIG. 8A is an exemplary side view of a drill bit.

FIG. 8B is an exemplary perspective view of a drill bit.

FIG. 8C is an exemplary bottom view of a drill bit.

FIG. 9a is an exemplary view of an implant.

FIG. 9b is an exemplary exploded view of an implant.

FIG. 10 is an exemplary flow chart describing the formation of an implant.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

Generally referring to exemplary FIGS. 1-8, a dental implant, system and method for using and providing anatomical dental implants may be described. A dental implant may be formed to substantially resemble a tooth which could have occupied a location corresponding to the location of an implanted dental implant. Additionally, one or more tools, such as a saw, drill and bit, Piezosurgery device and a laser such as a Er:YAG Laser may be used to provide one or more holes into which a dental implant may be implanted.

Exemplary FIGS. 1A and 1B may show exemplary front and side views, respectively, of a dental implant, for example a lower molar implant. Implant 100 may be such that it resembles a human molar which may be located or implanted in a mandible or lower human jaw. Additionally, implant 100 may be substantially smooth on its exterior portion which may be impregnated or otherwise treated with an osteogenic material similar to Hydroxyapatite and the like. Implant 100 may include a crown portion 102, which may be an artificial crown. Although crown portion 102 may be shown to resemble a molar in exemplary FIGS. 1A and 1B, it should be understood that any crown portion 102 may be designed, shaped or otherwise formed to appear similar to any desired tooth in a human mouth and may be formed in any desired manner. Further, implant 100, or any other implant described herein, may utilize any desired anchoring or securing implementation

Crown portion 102 may further be formed out of any desired material. For example, in some exemplary embodiments, crown portion 102 may be formed of an acrylic resin, hardened plastic, porcelain or any other known material used for forming artificial teeth. In still further exemplary embodiments, crown portion may be formed in any size or shape. In some exemplary embodiments, where crown portion 102 may be formed as a molar, crown portion 102 may be about 5 mm to about 8 mm in height about 5 mm to about 6 mm wide and have a depth of about 5 mm to about 6 mm.

In still further exemplary embodiments, implant 100 may include a root. A root may include platform 104. Platform 104 may be coupled to crown portion 102. Additionally, platform 104 may be formed so as to provide a desired contact and adhesion area with crown portion 102. For example, platform 104 may have a surface area of about 6 mm by about 6 mm, or approximately the same surface area as crown portion 102. Thus, as the dimensions of crown portion 102 may be varied in different examples where different sizes of crown portion 102 may be desired, an area of platform 104 may be varied or changed correspondingly. Additionally, platform 104 may have a height or depth of about 4 mm, or anywhere in a range of about 2 mm to about 4 mm, and may extend from a position coupled to or proximate to crown portion 102 to root portions 106 and 110.

Platform 104 may further include any number of root portions. In the exemplary embodiment shown in FIGS. 1A and 1B, an implant 100 with two root portions for example roots 106 and 110 may be shown. Root portions 106 and 110 may be formed in conjunction with platform 104 so as to allow for their formation out of a single piece of material. Alternatively, root portions 106 and 110 may be coupled to platform 104 in any desired manner. Additionally, roots 106 and 110 may be arranged in a substantially parallel manner with respect to each other.

In some exemplary embodiments, root portions 106 and 110 may be formed in any desired size or shape. For example, root portions 106 and 110 may be formed in substantially rectangular shapes or in shapes having a substantially rectangular body and a substantially elliptical distal end portion. Thus, in this example, roots 106 and 110 may be formed substantially similar to the roots of natural human molars and may provide benefits substantially similar to the benefits provided by natural teeth roots.

In further exemplary embodiments and referring still to FIGS. 1A and 1B, platform and roots 106 and 110 may be formed in any desired length and may be formed out of any desired material. For example, roots 106 and 110 may be about 10 mm to about 14 mm in length, with some exemplary embodiments measuring about 10 mm. As discussed with previous exemplary embodiments, the length of roots 106 and 110 may be varied as desired, for example to provide better anchoring in mouths or areas of different sizes. Roots 106 and 110 may be substantially rectangular and may have a width of about 3 mm and a depth of about 8 mm, although widths of about 1 mm to about 3 mm and depths of about 8 mm may be utilized, as desired. Additionally, in exemplary embodiments where the roots 106 and 110 have a width of about 3 mm and a depth of about 8 mm, there may be a gap disposed between root 106 and root 110 that is about 2 mm. This gap may be similar to an area found between the roots of a natural tooth. Also, in some other examples, roots 106 and 110 may be tapered or shaped in any other desired format, for example have a proximal portion that is wider than a distal portion or having a distal portion that is shaped in any desired format. Additionally, in some further exemplary embodiments, there may be a third root in addition to root 106 and root 110. In such exemplary embodiments, the dimensions of the roots may be varied as desired to provide a desired fit of the roots into a hole and a desired size of the tooth portion 102 in the mouth of a patient. Further, as with all exemplary embodiments described herein, any dimensions of implant 100, or any other implant or tool, may be varied, adjusted or changed as desired for a particular application. For example, as is known in the art, a different size implant may be needed for a patient due to potentially unique features or dimensions of a patient's mouth.

Additionally, roots 106 and 110, as well as platform 104 may be formed out of any desired material. For example, roots 106 and 110, as well as platform 104, may be formed out of commercially pure titanium, which may be of different grades or any desired alloy composition. In still further exemplary embodiments, roots 106 and 110 may be substantially smooth and have a coating, for example of calcite or hydroxyapatite coating. A hydroxyapatite or calcite coating, or any other desired coating, may be similar to that found naturally on the roots of teeth and thus may allow for benefits similar to the seating of a natural tooth in bone.

Still referring to FIGS. 1A and 1B, roots 106 and 110 may include end portions 108 and 112, respectively. End portions 108 and 112 may be formed as the same component of roots 106 and 110 as well as platform 104. End portions 108 and 112 may be curved or rounded and can further act to provide additional anchoring and security for implant 100 after it is implanted into the mouth of a patient. Additionally, in exemplary embodiments where three or more roots may be disposed on an implant 100, a number of end portions corresponding to the number of roots may be utilized.

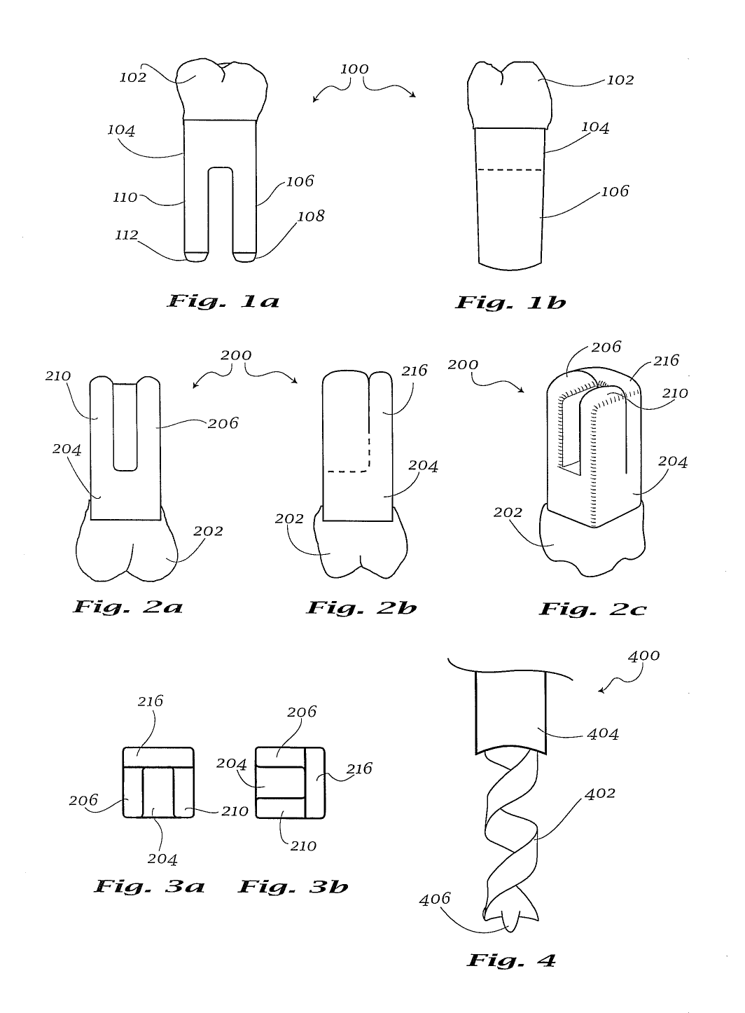

Now referring to FIGS. 2A, 2B and 2C, another exemplary dental implant 200 may be shown in a front view (FIG. 2A), a side view (FIG. 2B) and a perspective view (FIG. 2C) Implant 200 may be substantially similar to implant 100, however it may be designed to be implanted into the maxilla or upper jaw bone of a patient and may include a third root. Crown portion 202 may be formed in any desired size and shape, as with crown portion 102, and may have any desired dimensions. Additionally, crown portion 202 may be formed out of any desired material, for example an acrylic resin, hardened plastic, porcelain or any other desired material.

Similarly, implant 200 may include a platform 204 (similar to platform 104 of FIGS. 1A and 1B) and roots 206, 210 (which may be referred to as anterior or buccal roots) and 216 (which may also be referred to as a palatal root). Again, similar to the above exemplary embodiments discussing platform 104 and roots 106 and 110, platform 204 and roots 206 and 210, as well as any other roots, such as root 216, may be formed out of any desired material, for example commercially pure titanium, and may have any desired size, shape and dimensions, as desired for any application or fitment. For example, roots 206, 210 and 216 may have a substantially curved or elliptical end or distal portion and also may be tapered insofar as a proximal portion of any of roots 206, 210 and 216 may be wider than a distal portion of roots 206, 210 and 216, respectively. Additionally, platform 204 may have a width of about 3 mm to about 6 mm, for example about 4 mm. Also, while roots 206 and 210 maybe substantially similar to those described with respect to FIGS. 1A-B, root 216 may have an distal width of about 2 mm and a proximal width of about 5 mm. Additionally, root 216 may have a substantially parabolic or pyramidal shape, including a curved end portion, as described above. Additionally, and in FIG. 2C and as shown below with respect to FIG. 3, there may be a gap of about 1 mm, which may be varied as desired, between root 206 and root 216 and between root 210 and root 216.

Still referring to FIGS. 2A-C, and now also referring to the top-down view of exemplary FIGS. 3A and 3B, an exemplary implant 200 having three roots may be shown. In this example, roots 206 and 210 may be substantially described with respect to FIGS. 2A and 2B. Additionally, a third root 216, as shown in the front view of FIG. 3A and the side view of FIG. 3B, may be utilized with implant 200. In this exemplary embodiment, each of roots 206, 210 and 216 may be rectangular. Additionally, roots 206, 210 and 216 may each have a length of about 10 mm to about 12 mm, for example about 10 mm. Further, roots 206 and 210 may have a width of about 2 mm, and a depth of about 4 mm to about 7 mm, for example about 5 mm. Root 216 may have a width of about 2 mm and a depth of about 5 mm. Additionally, similar to the above description, each of roots 206, 210 and 216 may be coated with any material, for example a hydrophilic material, hydroxyapatite or any other nano-structure titanium alloy on the implant surface, which may provide it with many of the benefits of a natural tooth.

Additionally, as shown in the exemplary embodiments of FIGS. 3A and 3B, each of roots 206, 210 and 216 may extend from platform 204. Further, similar to the previous exemplary embodiments, the size, shape and orientation of the roots 206, 210 and 216 may be varied as desired. For example, in some examples, roots 206, 210 and 216 may have different lengths or roots 206 and 210 may share some or all dimensions while root 216 has different dimensions.

In some further exemplary embodiments, and referring to exemplary FIG. 8, a platform router bit may be used with an implant. Platform router bit 400 may include screw section 402, base section 404 and tip 406. Platform router bit 400 may have a width of about 6 mm and, in some examples, may be inserted, coupled or screwed-in about 4 mm, or any other depth as desired. Platform router bit 400 may then allow for the coupling of an implant or the securing of an implant in a desired location. Further, in some exemplary embodiments, platform router bit 400 may be used in conjunction with an oscillating saw, thus allowing for another manner of providing and inserting any of the implants described herein.

In some further exemplary embodiments, platform router bit 400, or any other type of bit, for example another type of drill bit, which is described in more detail below with respect to FIG. 8 may be coupled with a drill and utilize a square sleeve. The hole thus formed may be in the shape of a rectangle or may be substantially rectangular. A hole between about 3 mm and about 5 mm, for example about 4 mm, may then be drilled or bored into a desired area, for example the mandible or maxilla of a patient receiving an implant. The length and width of any hole drilled may correspond to a desired size of the dental implant, for example implant 100 or implant 200, which a patient may be receiving. In examples where a molar-style implant is to be implanted, platform router bit 400 may be used to provide a hole having a width of about 6 mm and a length of between about 5 mm and about 6 mm. The dimensions of the hole described herein are merely exemplary, however, and may be varied as desired. For example implants in the posterior regions of a mandible or maxilla may be larger than implants in anterior regions; therefore the size of a hole or holes for such implants may be correspondingly varied.

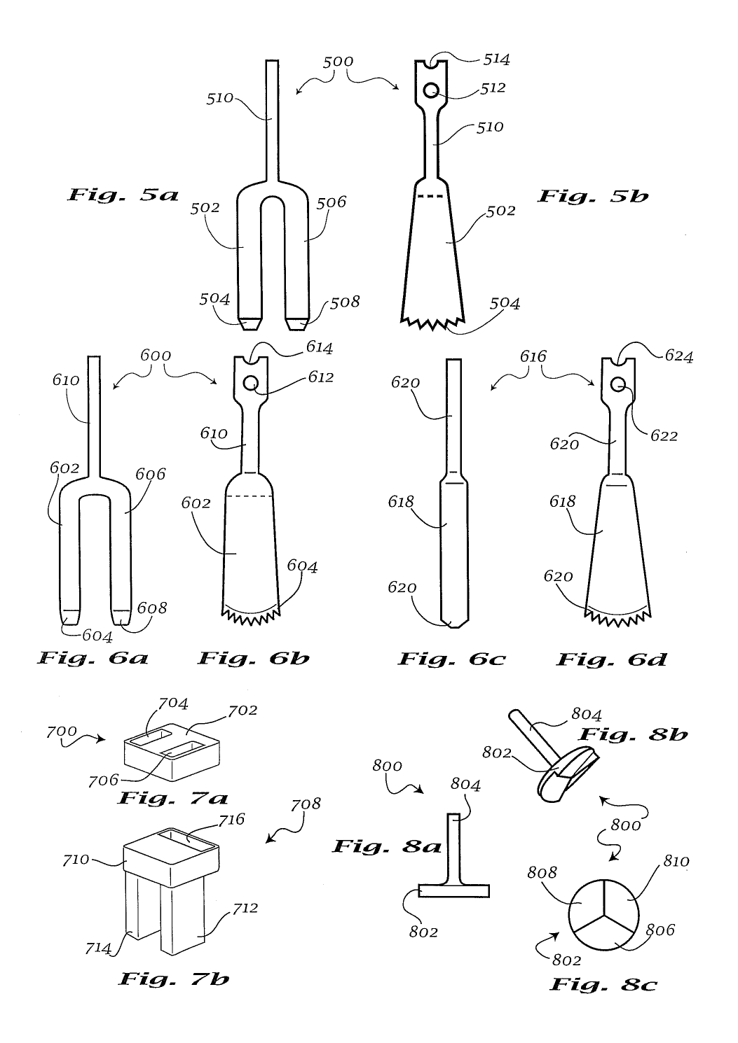

Following the preparation of the substantially rectangular hole as described above, a molar saw, such as those shown in FIGS. 5A-B and 6A-D, may be used to prepare areas for the root of the implant, such as those shown in FIGS. 1 and 2. Referring to FIGS. 5A and 5B, an exemplary lower molar saw 500 may be shown. Saw 500 may be about 21 mm in length and may include any number of saw arms, for example arms 502 and 506. Arms 502 and 506 may be coupled or formed with blades 504 and 508, respectively. Arms 502 and 506 may be formed out of any desired or known material so as to provide appropriate qualities when cutting or sawing. Additionally, arms 502 and 506 may be about 2 mm in width and may be separated by a gap of about 2 mm. Further, blades 504 and 508 may each be about 2 mm wide and may have a depth of about 4 mm. Also, saw 500 may include arm 510, which may be about 1 mm wide and about 10 mm long as well as eye 512 and notch 514, which may be used for coupling saw 500 to an actuating device or body. Thus, a total length of saw 500 may be about 16 mm to about 20 mm.

Now referring to FIGS. 6A, 6B, 6C and 6D, an exemplary upper molar saw 600 may be shown. Similar to lower molar saw 500, upper molar saw may be formed out of any desired material so as to provide appropriate qualities when cuffing or sawing. Additionally, saw 600 may be about 21 mm in length and may include any number of saw arms, for example arms 602 and 606. Arms 602 and 606 may be coupled or formed with blades 604 and 608, respectively. Arms 602 and 606 may be formed out of any desired or known material so as to provide appropriate qualities when cutting or sawing. Additionally, arms 602 and 606 may be about 1.5 mm in width and may be separated by a gap of about 1.5 mm. Further, blades 604 and 608 may each be about 2 mm wide and may have a depth of about 4 mm. Also, saw 600 may include arm 610, which may be about 1 mm wide and about 10 mm long as well as eye 612 and notch 614, which may be used for coupling saw 600 to an actuating device or body. Also, as further shown in FIG. 6A, in some exemplary embodiments, a blade 616 may be disposed between arm 602 and arm 606. Blade 616 may be about 2.5 mm in width.

In still further exemplary embodiments, any number of saws, for example two or more, may be used to prepare or bore three or more holes, as desired, for an implant. For example, two front two holes may be cut first, for example using a pair of saws formed similar to saw 600, after the square hole is cut for the platform, for example platform 204, of an implant. They can include two parallel saw blades 600 that are about 2 mm in thickness and about 4 mm to about 6 mm in width and in a range of about 10 mm to about 12 mm in length. The saws can be housed in a rectangular sleeve in guide 700, shown with respect to FIG. 7A, which can stabilize the saws and allow for precise placement. Sleeve 700 may have a body 702 and a pair of sleeves 704 and 706 through which a pair of saws or any other blades may pass and which. Guide 700 and sleeves 704 and 706 may then act as a guide for any sawing or cutting and, in some further exemplary embodiments, may be varied in size so as to allow for any desired saw or blade to be used as well as for any desired size of hole to be cut or otherwise formed.

After the first two holes are cut, a saw 616 of about 4 mm to about 6 mm in width, about 2 mm in thickness and about 10 mm and 12 mm in length can be used to create a back (palatal) hole. This saw can also be housed in a rectangular guide, for example guide 708. Guide 708 may include body 710, sleeve 716 and guide posts 712 and 714 that can fit down into the front two holes and allow for desired placement. The front two holes can join with the back hole, whereby the front two holes can be about 2 mm in width and about 4 mm to about 7 mm in depth. The back hole can be about 2 mm in width and about 5 mm in depth. There can also be a gap or space of about 2 mm between the front two holes. All three holes can be anywhere in a range of about 10 mm to about 12 mm in length joining with the platform hole that can be about 4 mm in depth. Further, as with guide 700, guide 708 may be formed in any size or shape as desired and each of guides 700 and 708 may be formed out of any desired material or materials.

In some further exemplary embodiments, and referring back to FIGS. 5A-B and 6A-D, saws 500, 600 and 616 may be formed in any desired size or shape and may include any number of desired interchangeable parts. For example, larger or smaller saws 500, 600 and 616, including larger and smaller blades, may be used as desired for different patients to create different size holes for different size implants. Additionally, the saws 500, 600 and 616 may be used at any location in a mouth, for example any location on a posterior or anterior maxilla or mandible and may be used to assist or benefit the implant of any types of teeth or other implants. Further, in some exemplary embodiments, a third arm or saw, or any number of saw or cutting blades may be attached or coupled to saws 500, 600 and 616. For example, if an implant having three roots were to be implanted into the mouth of a patient a saw having three blades may be used to saw or cut the desired holes.

In a further exemplary embodiment and still referring to both FIGS. 5A-B and 6A-D, saws 500, 600 and 616 may be inserted into a square hole, for example a hole previously bored by platform router bit 400 of FIG. 4. Saws 500, 600 and 616 can be connected to an actuating body (not shown) and then partially or fully shrouded or otherwise covered with a guide or sleeve, for example guide 700 or 708, as described above with respect to FIGS. 7A-B. In still further exemplary embodiments, the guide, for example guide 700 or 708, may be such that its dimensions correspond or substantially correspond with the substantially rectangular hole bored by drill bit 400, as described above. Additionally, the guide, for example guide 700 or 708, may be such that it can be positioned in a hole that has already been prepared or may be attached to the body of an actuator.

After saw 500 or 600 is coupled with an actuator, holes for the roots of an implant, for example roots 106, 110, 206 and 210 may be prepared. The actuator may be, for example, an oscillating or reciprocating saw body that is capable of moving blades 500 or 600 in a desired fashion so as to facilitate cutting or sawing. The holes that are prepared may be a substantially desired size for the roots of a corresponding implant. For example, if implant 100 is being fitted into the mandible of a patient, two holes may be prepared by saw 500. The holes may be any desired length, for example about 10 mm, about 12 mm, about 14 mm or any length from about 10 mm to about 14 mm. Additionally, the holes can be width, for example about 3 mm and may be any depth, for example about 8 mm. Further, additional holes may be prepared as desired.

In another example, using implant 200 as an upper molar implant, two or three holes may be prepared by saw 600 and, as desired, saw 616. The holes may be any desired length, for example about 10 mm, about 12 mm, about 14 mm or any length from about 10 mm to about 14 mm. Additionally, the holes for the anterior roots 206 and 210 can be any depth, for example about 5 mm to about 6 mm and may be any width for example about 2 mm. The palatal root 216 may have a width of about 2 mm and a depth of about 5 mm. Any holes formed for the anterior roots 206 and 210 can be any width but, in this example, they may be about 2 mm and their depth may be about 5 mm to about 6 mm.

Following the use of saws 500, 600 and 616, a recess having the desired shape and measurement characteristics may be formed as the site of an implant, for example implant 100 or 200. The implant 100 or 200 may then be seated in the recess at a desired time and the recess may be allowed to heal with the implant at any time thereafter. For example, in some exemplary embodiments, an implant may be tapped into the prepared recess or site and, a cover screw or healing abutment may then be used and assist in the healing process.

In another exemplary embodiment, and now referring to exemplary FIGS. 8A, 8B and 8C, a drill bit for a dental implant system may be described. The drill bit 800, which may be a lower platform bit, may be formed out of any material known in the art. Additionally, the bit 800 may have a width or diameter of about 6 mm and a bit depth of about 1 mm. Additionally, the bit 800 may include arm 804 which can be used to couple drill bit 800 to a drill body and may have a diameter, length and/or width of about 1 mm. In some other exemplary embodiments, a substantially similar bit may be used as an upper platform bit. As will be described in more detail below, the bit 800 can be housed in a rectangular sheet which can allow it to drill a hole approximately 4 mm in depth and having a length and width of about 5 mm to about 6 mm.

Still referring to FIGS. 8A-8C, drill bit 800 may have include drill bit head 802 that can include a number of segments, for example segments 806, 808 and 810. Segments 806 through 810 may be raised, offset or disposed in any of a variety of manners, for example as shown in FIG. 8B, so as to allow for a desired holed to be drilled or bored. In still further exemplary embodiments, drill bit 800 may be utilized with a sleeve or guide, such as that shown in exemplary FIGS. 7A-B or in accordance with any of the exemplary embodiments described herein.

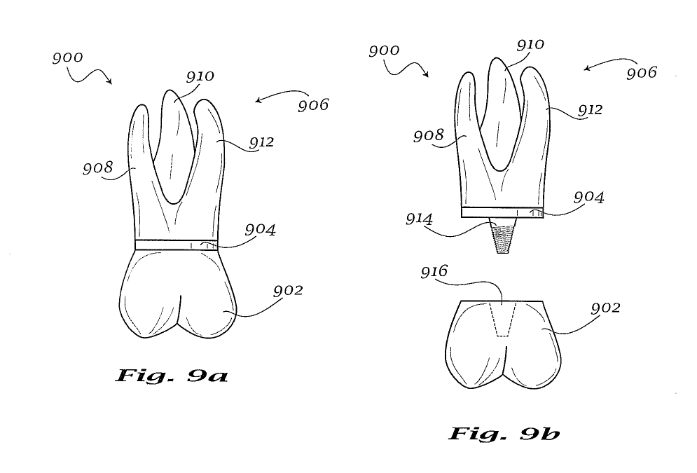

In yet another exemplary embodiment, and referring now to FIGS. 9a and 9b, an anatomical dental implant may be shown. In this example, implant 900 may have a crown or tooth portion 902, an abutment 904 and roots 906. Additionally, roots 906, such as root 908, root 910 and root 912 may be formed so as to resemble, either substantially or exactly, the roots of a tooth that is either extracted or desired to be extracted.

For example, roots 906 may be formed using materials substantially as described previously. However, roots 906 may be formed having shapes and sizes that substantially correspond to those of a tooth to be extracted or already extracted. Thus, implant 900 may be such that it fits substantially in an extraction site where a tooth having substantially similar or the same dimensions and characteristics was previously seated. In such an exemplary embodiment, after the implant 900 is inserted into an extraction site, the site may heal and accept the implant in a substantially natural manner, for example without any additional surgery or drilling.

In some further exemplary embodiments, any desired size and shape of implant 900 may be formed. For example, implant 900, and specifically roots 906, may be anatomically designed to meet the substantially exact dimension of the roots of a natural tooth. Different sizes of roots 906 can be formed to accommodate any desired individual characteristics of any desired tooth of a patient. Such designs of implant 900 may then be kept in stock by a dentist or oral surgeon to implant following the extraction of a natural tooth of a patient. For example, implant 900 could be placed in an extraction site at the time of the extraction. The size of the implant chosen may be determined by measuring the dimensions of the extracted tooth and matching it with an implant having substantially the same dimensions. For example, the roots of an extracted tooth may substantially be matched with the roots 906 of an implant 900 that have substantially the same size and location. As desired, after it is implanted, implant 900 can be stabilized with an internal screw device 914 inserted into receiving hole 916, and roots 906 may then be allowed to heal and integrate. Then abutment 904 can be added and the crown portion 902 may be mounted thereto.

Additionally, as shown in exemplary FIG. 9b, implant 900 may be formed in separate portions and later coupled. In this example, roots 906 may first be implanted into the mouth of patient where a previous tooth has been extracted. Then, at any desired time thereafter, crown portion 902 may be coupled to roots 906 through the abutment 904. In such an example, screw 914, or any other desired coupling device or mechanism, may be threadably inserted into receiving hole 916. Receiving hole 916 may extend through abutment 904 and into roots 906. This may allow for the coupling of crown portion 902 with implant 900 and, in particular, roots 906. Additionally, screw 914 can provide for additional stability.

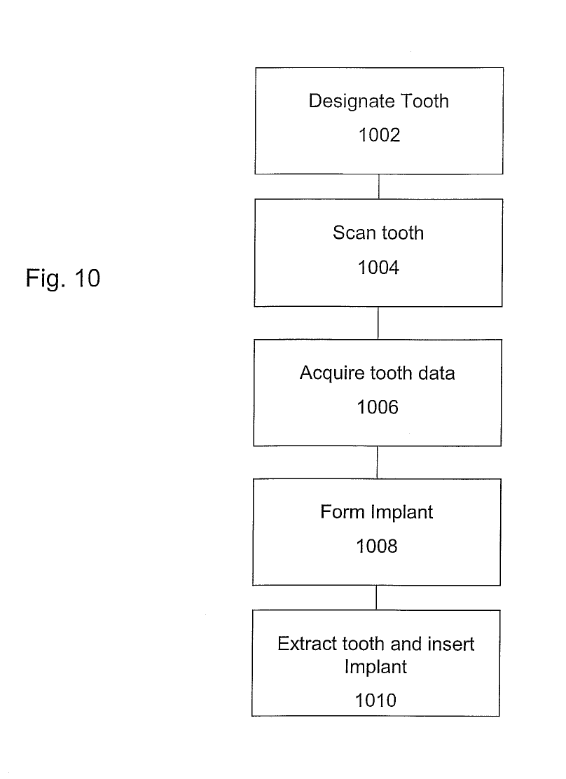

In a further exemplary embodiment, implant 900 may be formed as shown in exemplary FIG. 10. In this example a tooth from a patient may be substantially replicated so that after the tooth is extracted, an implant with substantially the same dimensions will be placed in the extraction site and allowed to heal substantially naturally. Thus, in 1000, a tooth to be extracted and replaced with an implant may be designated. The tooth may be designated for extraction for any desired reason. Then, in 1002, a three dimensional image of the tooth to be extracted may be generated and precise measurements and data of any desired elements of that tooth may be taken. For example, it may be desired to obtain data regarding the size and shape of the roots of the tooth that is being extracted, as well as the dimensions of the roots, as well as any other characteristics, for example the size, shape and appearance of the crown of the tooth to be extracted In some exemplary embodiments, the three dimensional image of the tooth and related data may be obtained through the use of a computed axial tomography (“CAT” or “CT”) scanner, for example a cone beam CT. Additionally, CAD-CAM technology and virtual imaging software may be used to determine and record any desired dimensions of the tooth, for example the cementoenamel junction (“CEJ”), the root itself, and apex of the root, as well as any others. Using this information, in 1004, an anatomical dental implant may be formed.

Using the data collected in 1004, an anatomical dental implant may be formed using additive manufacturing for rapid prototyping. This process can take virtual designs from computer aided design (CAD) and transforms them into thin, virtual, horizontal cross-sections and then creates successive layers until a model is complete. Thus, using this process, a virtual model can be formed substantially identically to a physical model. Further, with additive manufacturing, a machine can read data from a CAD drawing and lay down successive layers of material, for example, but not limited to, titanium, and build a custom implant from a series of cross sections. These layers, which can correspond to a virtual cross section from the CAD model, may be fused together automatically to create the final implant shape. The standard data interface between CAD software and the machine is typically an STL file format because smaller facets produce a higher quality surface, although it is contemplated that any other known format may be utilized. The anatomical dental implant may have the same dimensions as the tooth designated to be extracted. Thus, an anatomical dental implant can be formed to which an abutment and crown may later be coupled.

Still referring to exemplary FIG. 10, in 1006, the tooth designated to be extracted may then be extracted. Next, in 1008, the anatomical dental implant may then be implanted at the extraction site. This can be done at any time following the extraction of the designated tooth, for example about immediately after that tooth is extracted. As anatomical dental implant will have the same dimensions as the tooth that was previously extracted, it may fit substantially naturally in the extraction site. Depending on need or as desired, the anatomical dental implant may be implanted at the extraction site. The implant can be then be allowed to integrate into the mouth and the extraction site can be allowed to heal. Additionally, in some embodiments, an internal screw device can be used to stabilize the implant in the extraction site. Then, as desired, an abutment and crown may be coupled to the anatomical dental implant.

Referring generally to exemplary FIGS. 9 and 10, any portions of anatomical dental implant 900 may be formed as desired. For example, with front teeth, an abutment 904 may be formed in such a manner as to be substantially or completely concealed when a crown or tooth portion 902 is coupled thereto. Additionally, with some teeth, the variations in size, shape and location of the roots may be such that they can be quickly and easily replaced using stock pieces. For example, from a central incisor back to a first bicuspid, enough variations on the size and shape of the tooth may be made and kept in stock such that after a tooth is extracted, it may be measured, for example by calipers or computer imaging, and an appropriate anatomical dental implant may be chosen from a stock selection.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. An implantable dental apparatus, comprising:
an artificial crown;
a base portion coupled to the artificial crown; and
at least a first substantially anatomically-shaped root projecting from the base portion.

2. The implantable dental apparatus of claim 1, further comprising at least a second substantially anatomically-shaped root projecting from the base portion.

3. A method for forming an anatomical dental implant, comprising:
designating a tooth to extract and replace with an implant;
generating a three dimensional image of the tooth;
obtaining the dimensions of the tooth from the three dimensional image;
forming a mold based upon the dimensions of the tooth;
forming an implant anatomically shaped and sized substantially identically to at least a root section of the designated tooth.

4. The method of claim 3, wherein the three dimensional image of the tooth is made with a computed axial tomography (“CT”) scanner.

5. The method of claim 4, wherein the CT scanner is a cone beam CT scanner.

6. The method of claim 4, further comprising forming an implant anatomically shaped and sized substantially identically to the roots of the tooth.

7. The method of claim 4, further comprising forming an implant anatomically shaped and sized substantially identically to the entire tooth.

8. The method of claim 3, further comprising extracting the designated tooth and implanting the anatomical dental implant in a location from where the designated tooth was extracted.

9. The method of claim 8, further comprising integrating roots of the anatomical dental implant into the location from where the designated tooth was extracted.

10. A system for anatomical dental implants, comprising:
a designated tooth to be extracted;
a cone beam CT scanner that generates a three dimensional image of the tooth to be extracted;
virtual imaging software that analyzes the three dimensional image of the tooth to determine dimensions of the tooth; and
a mold created based upon the dimensions of the tooth and used to form an anatomical dental implant.

11. The system of claim 10, wherein the virtual imaging software includes CAD-CAM technology.
